(19) United States
(12) Patent Application Publication
(10) Pub. No.: US 2012/0029563 A1
(43) Pub. Date: Feb. 2, 2012

(54) METHOD AND SYSTEM FOR TREATING PATIENTS

(76) Inventor: Andrew Swanson, Kingston, WA (US)

(21) Appl. No.: 13/192,424

(22) Filed: Jul. 27, 2011

Related U.S. Application Data
(60) Provisional application No. 61/368,231, filed on Jul. 27, 2010.

(51) Int. Cl. A61F 5/00

(52) U.S. Cl. 606/237

(57) ABSTRACT

There is provided a system and method for treating a patient by administering a predetermined sequence of adjustments to a patient's body. Unlike the unstructured or random application of adjustments used in prior art chiropractic treatments, the treatment sequences and provided for herein unlock function of dysfunctional joint systems in a manner analogous to a combination lock mechanism. Disciplines that may utilize embodiments of the present invention include chiropractic, naturopaths, sports medicine, physical therapy, professional and collegiate trainers, as well as aspects of veterinary medicine. Through applications of the method of the present invention, patients may receive a predetermined number of treatment applications, and have long-lived or substantially permanent results from the treatment without the need for ongoing adjustments to spine or other body structures.

### CROSS REFERENCE TO RELATED APPLICATIONS

This application claims the full benefit and priority of U.S. Provisional Application Ser. No. 61/368,231, titled “Method and System for Treating Patients” filed on Jul. 27, 2010, the disclosure of which is fully incorporated by reference herein for all purposes.

### BACKGROUND

1. Field of the Invention

The present invention relates to systems and methods for treating patients by administration of a predetermined sequence of physical manipulations to the patient's body. More particularly, the present invention provides long-term resolution of symptoms by correcting movement dysfunctions of certain joints in the patient's body, including manipulation of a predetermined sequence of joints in the patient's extremities. Disciplines that would be inclined to utilize embodiments of the present invention include chiropractic, naturopaths, sports medicine, physical therapy, professional and collegiate trainers, as well as applications to veterinary medicine.

2. Description of the Related Art

Since the late 1800's, the chiropractic health care discipline has provided treatment options to patients to address a wide variety of disease processes and neuromusculoskeletal conditions. The treatment has often focused on correcting “subluxations” through a variety of manipulation techniques, some of which may be performed entirely by controlled administration of force by the chiropractic physician, and others through assistance of certain mechanical and/or electrical devices. As defined by the World Health Organization, a chiropractic subluxation constitutes “a lesion or dysfunction in a joint or motion segment in which alignment, movement integrity and/or physiological function are altered, although contact between joint surfaces remains intact. It is essentially a functional entity, which may influence biomechanical and neural integrity.”

Common chiropractic patient management involves spinal manipulation and other manual therapies to the joints and soft tissues. Spinal manipulation, which chiropractors may also call “spinal adjustment” or “chiropractic adjustment,” is the most common treatment used in chiropractic care to remove nerve interference, restore patient overall health, and also relieve pain. Complementary treatments may also include rehabilitative exercises, health promotion, electrical modalities, complementary procedures, and lifestyle counseling.

An array of diagnostic methods and treatment techniques were developed in the chiropractic profession to identify and correct chiropractic subluxations. Popular chiropractic treatment methods include: Diversified, Gonstead, SOT, Motion Palpation, Applied Kinesiology, Activator Method, Grostic, DNFT, Atlas Orthoginal, and Toftness. Some techniques start from the upper spine and work towards the lower spine, others from the lower spine to upper spine. Some focus on the upper and others focus on the lower spine. All chiropractic techniques involve random treatments of the spine.

Traditional chiropractic correction of the subluxation with random techniques usually involves a lengthy process of repetitive treatment which requires patients to receive regularly scheduled adjustments for months at a time. Patients are informed that since most conditions arise over long periods of deterioration, an extended treatment will program will be needed to regain the “momentum” required through repeated adjustments to alleviate the body's tendency to return to its subluxated state.

Patient compliance issues led to the formation of practice management companies designed to produce successful doctors who have learned specific methods to keep patients focused on their lengthy treatment programs. Testimonials of doctors who have doubled their monthly incomes with such patient management techniques can be found in most chiropractic newspapers and magazines. These programs emphasize that if the chiropractic physician can manage patients and keep them on their programs, the physician can be successful and wealthy.

Trust issues have surfaced as patient compliance became more the focus for some practitioners in the chiropractic profession, rather than patient welfare. Insurance companies have become less tolerant of treatment standards in the chiropractic discipline, and physical therapists are challenging the expertise of chiropractic physicians. Association with other health care professions such as allopathic medicine needs to be strengthened. Patients can become disenchanted with the costs associated with repetitive lifelong treatment, and with the extended treatment time needed to address health issues. The chiropractic profession is suffering from these and other major challenges. If the approach to chiropractic care were more streamlined and more efficient, both the patient and the chiropractic profession would benefit. For this to happen, a new model is needed.

Therefore, what is needed is a chiropractic treatment system that provides for expedited patient healing with a predicable number of treatments. What is also needed is a chiropractic treatment method that provides for reduction of sports injuries through coordinated adjustment of body structures. What is also needed is a method to enhance freedom of motion in motion-absorbing joints and components of the body to promote wellness and provide for long-term treatment and prevention of subluxations.

### SUMMARY OF THE INVENTION

There is provided a system and method for treating a patient by administering a predetermined sequence of adjustments to a patient's body. Unlike the unstructured or random application of adjustments used in prior art chiropractic treatments, the treatment sequences and provided for herein unlock function of dysfunctional joint systems in a manner analogous to a combination lock mechanism. Disciplines that may utilize embodiments of the present invention include chiropractic, naturopaths, sports medicine, physical therapy, professional and collegiate trainers, as well as aspects of veterinary medicine. Through applications of the method of the present invention, patients may receive a predetermined number of treatment applications, and have long-lived or substantially permanent results from the treatment without the need for ongoing adjustments to spine or other body structures. In some instances, after receiving treatment, patients that continue to walk on hard, flat surfaces (which most patients will routinely encounter) may still benefit from periodic treatment of the foot/ankle areas.

Each day, many people walk an average 10,000 steps. Each step produces a ground reactive force of approximately 110% of body weight. This force is coupled through the foot and transmitted up the leg at approximately 200 miles per hour. The average amount of force a 200 pound individual would encounter in one day is estimated at 640 metric tons, or 700 U.S. tons.

The study of the human gait cycle shows that beginning with heel strike, feet adapt to the ground surface walked upon, and likewise, the feet absorb the shock of each step. This absorbing of shock slows down the ground reactive force so the surrounding tissues of the leg can further dampen it. The result is reduced stress on the musculoskeletal system.

The absorption of force is dependant on proper joint function throughout the foot and ankle. These joints must be able to move within their ranges to efficiently absorb shock. Unfortunately, the average foot and ankle joints do not move within their ranges, since they experience an abundance of joint dysfunction.

This joint dysfunction is caused, at least in part, by the nature of modern-day walking surfaces. Before paved walkways and solid flooring were commonplace, humans used to walk on irregular surfaces such as dirt, sand, moss, rocks, and tree roots. The normal deformation of the feet when traversing these uneven surfaces promoted more flexibility in the joints of the foot and ankle while walking. Additionally, natural surfaces were often more cushioned, which further reduced the stress coupled to the human body from the process of walking.

The surfaces we now walk on, concrete, asphalt, hardwood, tile and marble, are less forgiving and more rigid. These flat, hard surfaces do not promote joint flexibility, rather they promote joint dysfunction. Without proper joint function the foot and ankle are unable to efficiently absorb and dissipate ground reactive forces. This leads to pathological amounts of stress repeatedly travelling throughout the musculoskeletal system. Further, in an attempt to stabilize itself, the spine tightens muscles and compresses joints to adapt to this continuous force. The result is a degenerating spine that lacks function and mobility, creating an environment for injury.

It is known in the literature that joints in the sacroiliac region react to oncoming force by compression of the sacrum on the ileum. This takes place through a series of surrounding muscle contractions compressing the sacroiliac joint together, as the body “braces” for impact. Studies suggest that the body attempts to compress the joints together to enhance stability.

Underlying theories of the present invention are similarly founded on the following principles: modern-day humans are surrounded by an abundance of flat, hard, and mostly horizontal walking surfaces. Walking and running in this environment causes the joints in the feet and ankles to become dysfunctional. As a result, we can no longer absorb the shock from ground reactive forces efficiently. Every day, just from walking and related activities, approximately 700 tons of unimpeded force travel up legs, into the pelvis and spine. In an attempt to brace itself from repetitive heel strikes and the generated force, the human body compresses joints together throughout the musculoskeletal structure to provide stability. This compression of joints involves the feet, ankles, knees, hips, pelvis, spine, shoulders, elbows and wrists.

In the present model of chiropractic, in prior art approaches a practitioner will examine a new patient's spine and determine the presence of multiple joint dysfunctions, or subluxations. Random adjustments will be administered to the patient's spine based on a specific or combination of chiropractic techniques the particular doctor practices. The patient will leave the clinic and return to the surrounding “hardscape” which promotes further bracing and joint compression. It is with little surprise that the following day, the patient returns for treatment with the same joint dysfunction as before. The traditional reasoning of the chiropractic profession is repetitive, long term treatment is necessary to correct a condition which has been long standing.

In contrast, methods and systems of the present invention address the underlying issues that arise from the patient's traversal of surrounding “hardscape” surfaces. In one embodiment, joint dysfunctions within the gait cycle can be corrected to allow for more efficient transfer of forces, less bracing, and reduced joint compressions. For example, when normal motion is restored to the foot, the improved strike force handling of body structures allows the spine to flex and operate normally again, restoring normal nerve supply and joint function throughout the body. Methods of the present invention were developed to restore normal function by applying certain treatments in a specific order. The developed methods and systems of the present invention will allow the patient's body to respond favorably to a “pattern” of adjustments with an automatic correction of joint dysfunction. In a preferred embodiment, the patterns that are utilized by the practitioner treat the body systems in the order that follows the natural shockwave that propagates from the striding foot impacting the ground and travels up the leg through the pelvis and into the opposite body side in the thoracic, arm, and upper body regions. Treatment of the body systems related to the first stride impact is referred to herein as the “primary treatment cycle.” The second leg and upward body structures is treated in a similar manner in the order of shockwave propagation as the shockwave were to propagate from the second impacting foot into the body. Treatment of the body systems related to the second foot stride is referred to herein as the “secondary treatment cycle.” In a preferred embodiment, the patient's dominant leg (or dominant side) is determined from a pre-treatment evaluation, wherein the primary treatment cycle begins with the dominant foot/leg, and once the primary cycle is complete, the secondary treatment cycle begins with the non-dominant foot/leg and is applied upwards into the related body structures. In the preferred embodiments, patterns of adjustments commonly produce a permanent or very long-lasting correction of joint dysfunction in various areas of the patient's body.

In traditional chiropractic practice, physicians are taught various methods to adjust the upper thoracic region in their patients. Subluxations are commonly present in the spine at levels T1-T2, T2-T3, T3-T4. Using Gonstead and Diversified techniques, these joints can be mobilized with the practitioner's thumb pushing on the individual vertebrae. This is called a “thumb move.” This area can also be mobilized by pushing with the pisiform in the heel of the hand. This referred to as the “modified diversified pisiform.” These random approaches are very effective in restoring joint function to the upper thoracic region. However, the corrections are temporary and require repetitive treatments which offer no permanent benefit.

Embodiments of the present invention make persistent corrections to various body structures without direct physical manipulation of those body structures. For example, application treatment modalities of embodiments of the present invention allow the body structures to work together to achieve comprehensive readjustment, such as the self-adjustment of the thoracic spine without direct manipulation or treatment of the thoracic spine. A preferred embodiment of the present invention provides successful and persistent treatment results in 18 treatment steps consisting of a primary treatment cycle followed by a secondary treatment cycle; however, the practitioner may make adjustments to the treatment cycles to achieve the desired results in fewer or more treatment steps. On average, with the 18-step treatment approach, patients report substantial relief from symptoms, and such reports include favorable resolution of adverse symptoms related to: back and neck; hip and knee, foot, shoulder pain; tennis elbow; carpal tunnel; hamstring and groin injuries; headaches; migraine headaches; dizziness; disc problems; plantar fasciitis; Achilles tendonitis; and overall health. Further, treatment methods of the present invention assist patients with avoiding or delaying hip or knee replacement surgery when a diagnosis of joint degeneration has been made.

APPLICATION TO SPORTS. Treatment methods and systems of the present invention increase athletic ability by restoring normal joint mechanics, muscle and nerve function. Further embodiments of the present invention help treat and prevent common injuries that limit athletic performance, such as muscle strains of the groin and hamstring, as well as injuries to the plantar fascia and Achilles tendon. When a joint loses function, its corresponding muscle attachment (groin or hamstring, for example) becomes strained by the motion of surrounding joints. Once optimal function is restored to the affected joint through embodiments of the present invention, muscle performance exceeds the physical demands required in competitive sports activities.

One application of treatment methods of the present invention aids in reducing injury to both the hamstring and groin muscles during sporting activities by improving pubic symphysis mobility. In the human body, the respective ends of the hamstring and groin muscles are attached to the pelvis next to the pubic symphysis. The pubic symphysis is located in the front of the pelvis, behind the pubic area. The other respective ends of each muscle attach to the leg. When the pubic symphysis becomes locked or otherwise immobile, the corresponding muscles no longer track with the moving leg, which makes the muscles more susceptible to injury. Restoring normal motion to the pubic symphysis through methods of the present invention operate to decrease the occurrence of hamstring and groin injuries.

In a normally functioning pubic symphysis, the hamstrings and groin muscles will move together with the leg and the pelvis. If the pubic symphysis is locked or otherwise not functioning properly, the pubic bone is incorrectly anchored and causes improper motion of the leg, and problems arise with the attachment of the groin and hamstring muscles. In the case of a dysfunctional pubic symphysis joint, the hamstring and groin are only moving where they are attached to the leg, and in sports activities, where there is freedom of movement of the leg but improper coordinated movement of the pelvis, issues arise that may lead to injury such as hamstring and groin pulls and tears. While chiropractic practitioners have in the past performed adjustments to the pubic symphysis, mostly such adjustments resulted in the joint becoming re-locked in as soon as one day. However, it was found that a coordinated and structured set of adjustments, including the lower extremities, resulted in a longer lasting or permanent correction of pubic symphysis joint dysfunction. As provided herein, methods and systems of the present invention serve to prevent sporting injuries by restoring proper coordinated movement of the hip and leg through structured adjustments that provide long-term mobilization of the pubic symphysis.

In a preferred embodiment, a treatment method comprises administering to a patient a sequence of treatment steps in a primary treatment cycle, each of the treatment steps respectively comprising one or more treatment patterns, the treatment patterns respectively comprising one or more physical manipulations of body structures by a health care practitioner executed in a predetermined order; administering to a patient a sequence of treatment steps in a secondary treatment cycle, each of the treatment steps respectively comprising one or more treatment patterns, the treatment patterns respectively comprising one or more physical manipulations of body structures by a health care practitioner executed in a predetermined order; and wherein the sequence steps of the primary treatment cycle are applied starting from a bottom side of the dominant side of the body and moving toward the patient's upper body non-dominant side; and the sequence steps of the secondary treatment cycle are applied starting from a bottom side of the non-dominant side of the body and moving toward the patient's upper body dominant side. The dominant side of a patient's body may be determined by the practitioner before administering treatment, and may be performed by any desired method such as positioning the patient in a supine position with legs extended, alternatively moving the patient's knees towards the patient's chest, and monitoring the range of motion during the movement, and determining the dominant side of the patient corresponds to the side of the patient where the patient's leg encountered the most restrictive range of motion.

The sequence of treatment steps may be applied in any desired order. In a preferred embodiment, the sequence of treatment steps in the primary treatment cycle comprises sequential treatment of the patient's dominant-side foot, dominant-side knee, dominant-side hip, dominant-side pubic symphysis, dominant-side lower sacrum and upper ilium; non-dominant-side upper sacrum and upper ilium, non-dominant-side proximal clavicle, non-dominant-side shoulder, non-dominant-side elbow, and non-dominant-side wrist. In another embodiment, prior to the treatment of the patient's non-dominant-side proximal clavicle in the primary treatment cycle, the patient's dominant-side sacrotuberous ligament is treated with Logan-Basic Technique then the patient's non-dominant-side sacrotuberous ligament and/or long dorsal ligament is treated with with Logan-Basic Technique.

In a preferred embodiment, the sequence of treatment steps in the secondary treatment cycle comprises sequential treatment of the patient's non-dominant-side foot, non-dominant-side knee, non-dominant-side hip, non-dominant side pubic symphysis, non-dominant-side lower sacrum and upper ilium, non-dominant-side upper sacrum and upper ilium, dominant-side proximal clavicle, dominant-side shoulder, dominant-side elbow, and non-dominant-side wrist. In an alternate embodiment, prior to the treatment of the patient's dominant-side proximal clavicle, patient's non-dominant-side sacrotuberous ligament is treated with Logan-Basic Technique, then the patient's dominant-side sacrotuberous ligament and/or long dorsal ligament are treated with Logan-Basic Technique.

Methods of the present invention may execute treatment steps on any desired schedule. For example, in a preferred embodiment, each of the treatment steps of the primary treatment cycle are administered to the patient no more often than one treatment step per day, and each of the treatment steps of the secondary treatment cycle are administered to the patient no more often than one treatment step per day.

The number of treatment steps per treatment cycle may be devised by the practitioner to satisfy any desired treatment goal; for example, in one embodiment nine treatment steps are administered to the patient in the primary treatment cycle and nine treatment steps are administered to the patient in the secondary treatment cycle, and a total treatment sequence may include any desired number of treatment steps such as 17, 18, or 20 treatment steps. Preferably, only one treatment steps is performed on the patient in any calendar day, but as desired to meet a treatment goal such as total treatment length or sufficiency of unlocking joints, a plurality of treatment steps may be performed on the same calendar day. Through embodiments of the present invention, the patient is treated without applying direct treatment manipulation to one of the cervical spine or the thoracic spine, yet these structures of the patient's body are unlocked through the body's own response to the applied treatment sequences.

Another embodiment includes a method for treating subluxations in a joint of an organism, the method comprising providing adjustments to structures in a dominant foot of the organism, followed by a non-dominant foot of the organism, whereby improved motility in the foot structures mitigate shock impulses that are coupled to the joint from the feet when the feet strike a hard surface. In yet another embodiment, a method for reducing injuries in a living organism comprises providing adjustments to structures related to a foot and leg of the organism thereby improving flexibility of a public symphysis joint in the organism.

### BRIEF DESCRIPTION OF THE DRAWINGS

FIG. 1 illustrates a partial skeletal structure of a patient being treated with methods of the present invention, with the following treatment sequence locations identified: Sequence 1, Dominant-side Foot; Sequence 2, Dominant-side Knee, Sequence 3, Dominant-side Hip; and Sequence 4, Dominant-side Pubic Symphysis.

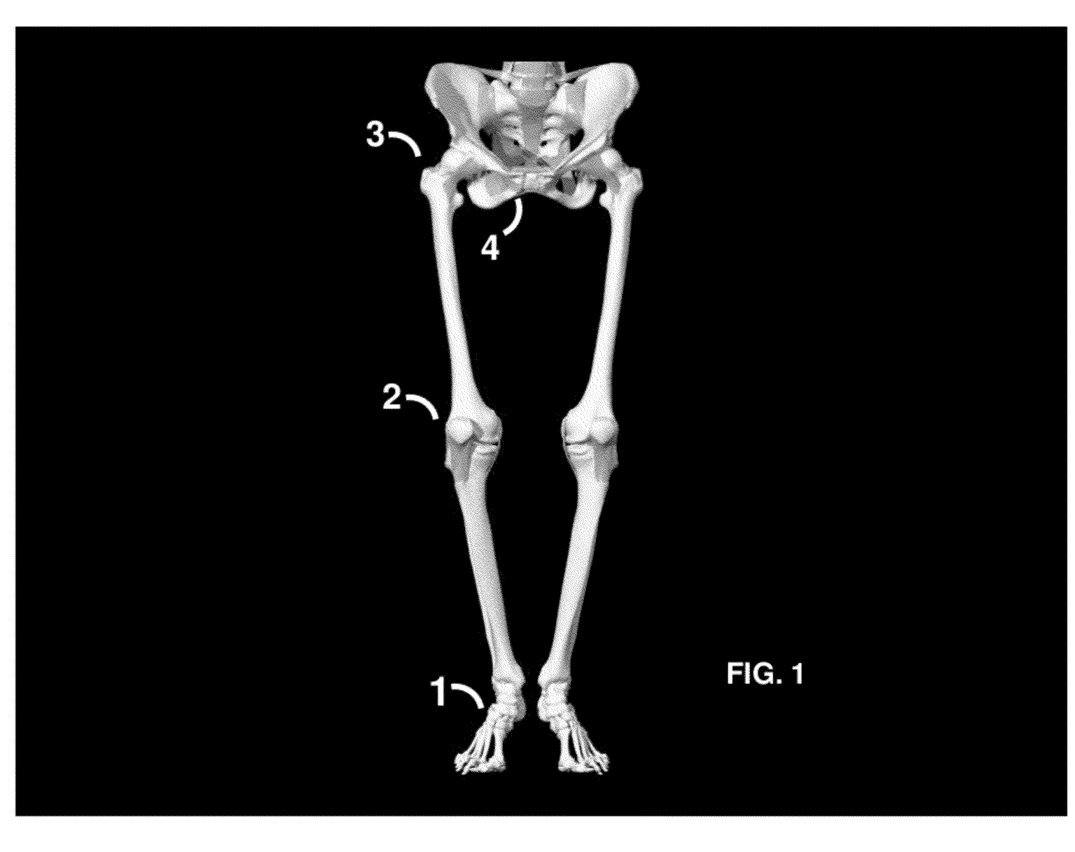

FIG. 2 illustrates a partial skeletal structure of a patient being treated with methods of the present invention, with the following treatment sequence locations identified: Sequence 5, Dominant-side Lower Sacrum and Upper Ilium; and Sequence 6, Non-Dominant-side Upper Sacrum and Upper Ilium.

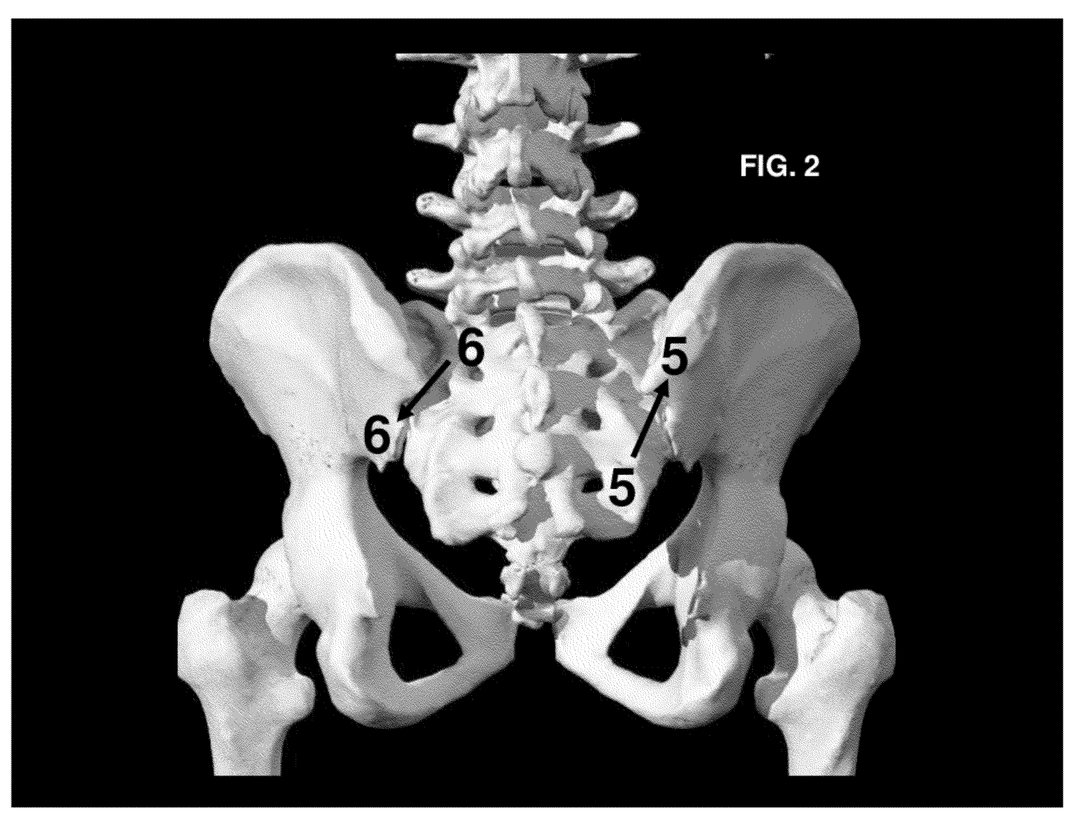

FIG. 3 illustrates a partial skeletal structure of a patient being treated with methods of the present invention, with the following treatment sequence locations identified: Sequence 6 (continued) Non-Dominant-side Proximal Clavicle; Sequence 7, Non-Dominant-side Shoulder; Sequence 8, Non-Dominant-side Elbow; Sequence 9, Non-Dominant-side Wrist.

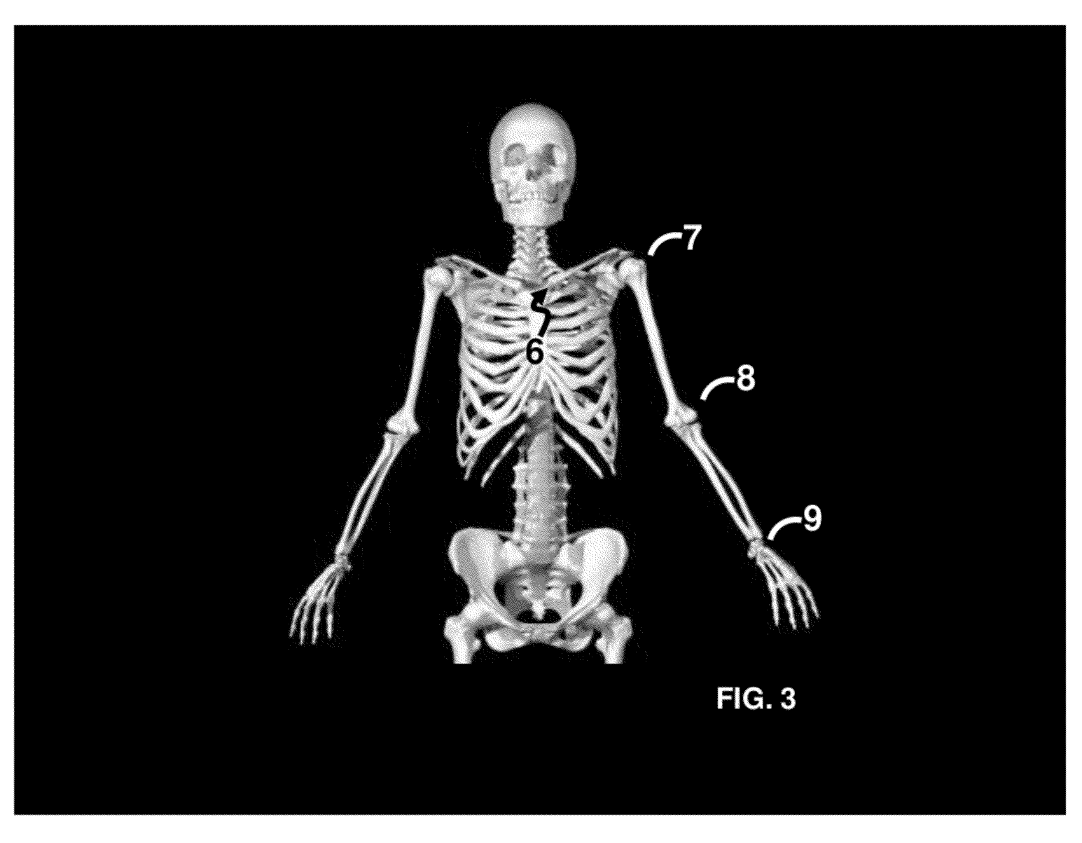

FIG. 4 illustrates a partial skeletal structure of a patient being treated with methods of the present invention, with the following treatment sequence locations identified: Sequence 10, Non-Dominant-side Foot; Sequence 11, Non-Dominant-side Knee, Sequence 12, Non-Dominant-side Hip; and Non-Sequence 13, Non-Dominant Side Pubic Symphysis.

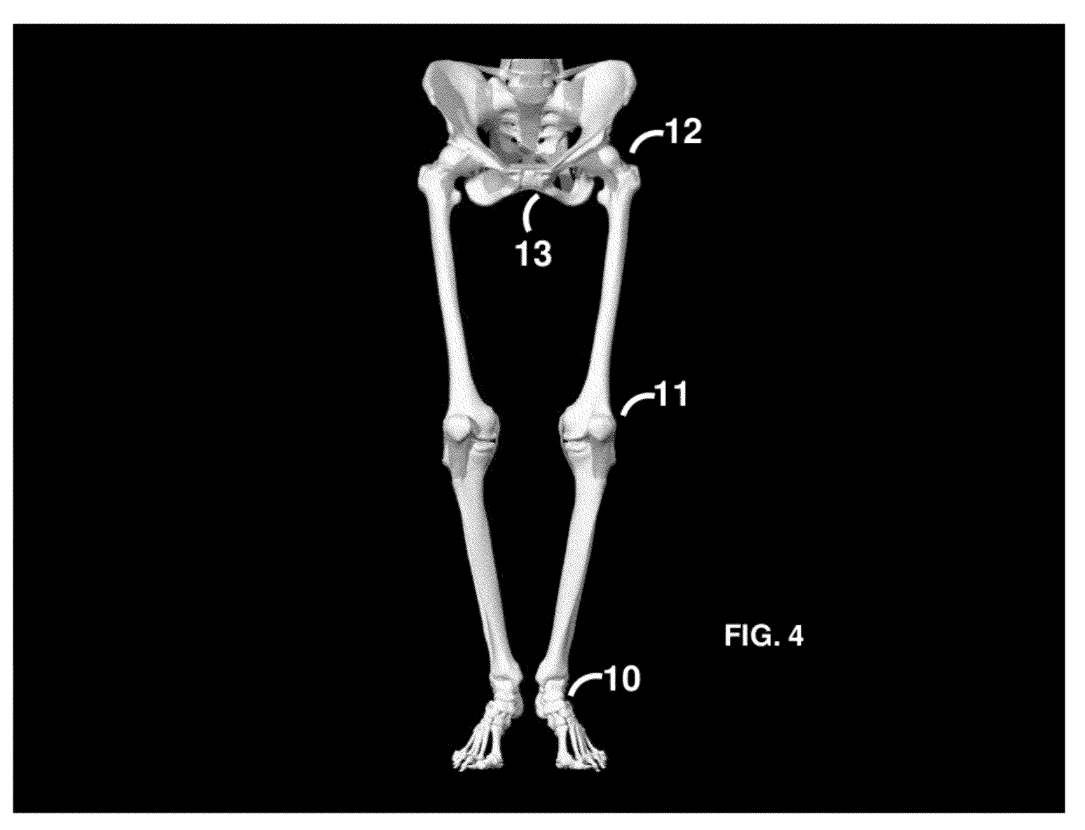

FIG. 5 illustrates a partial skeletal structure of a patient being treated with methods of the present invention, with the following treatment sequence locations identified: Sequence 14, Non-Dominant-side Lower Sacrum and Upper Ilium; and Sequence 15, Non-Dominant-side Upper Sacrum and Upper Ilium.

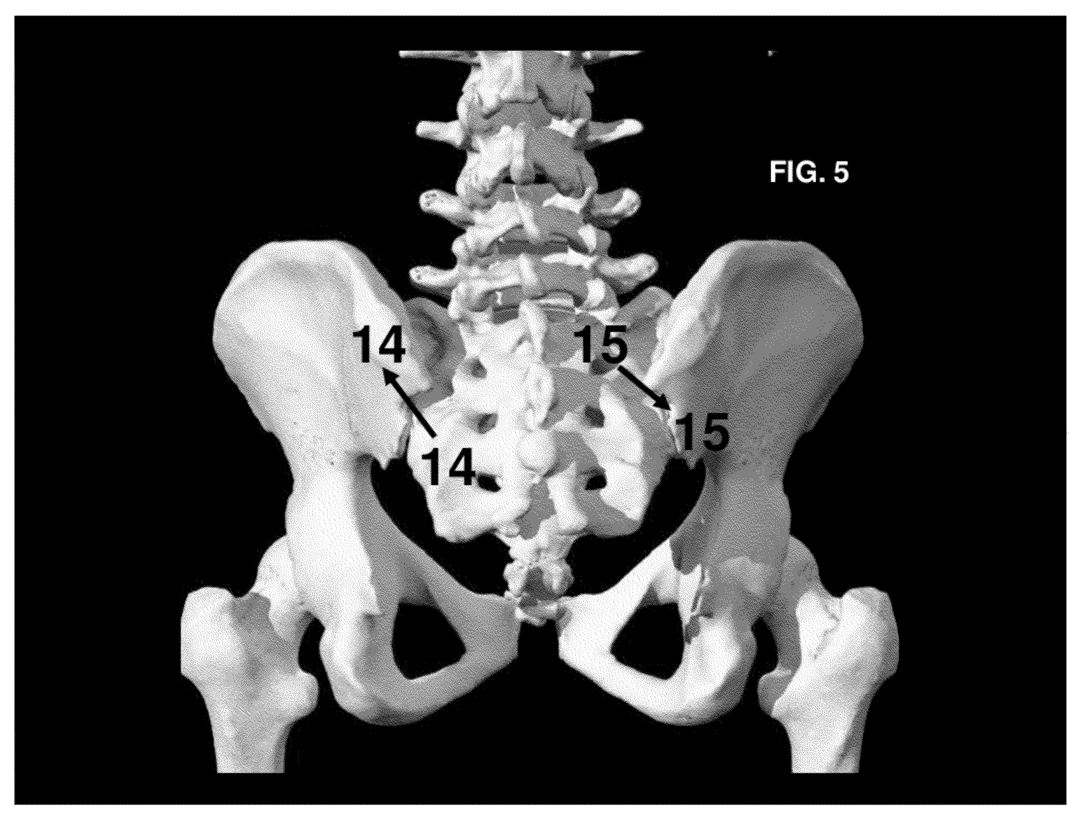

FIG. 6 illustrates a partial skeletal structure of a patient being treated with methods of the present invention, with the following treatment sequence locations identified: Sequence 15 (continued) Dominant-side Proximal Clavicle; Sequence 16, Dominant-side Shoulder; Sequence 17, Dominant-side Elbow; Sequence 18, Non-Dominant-side Wrist.

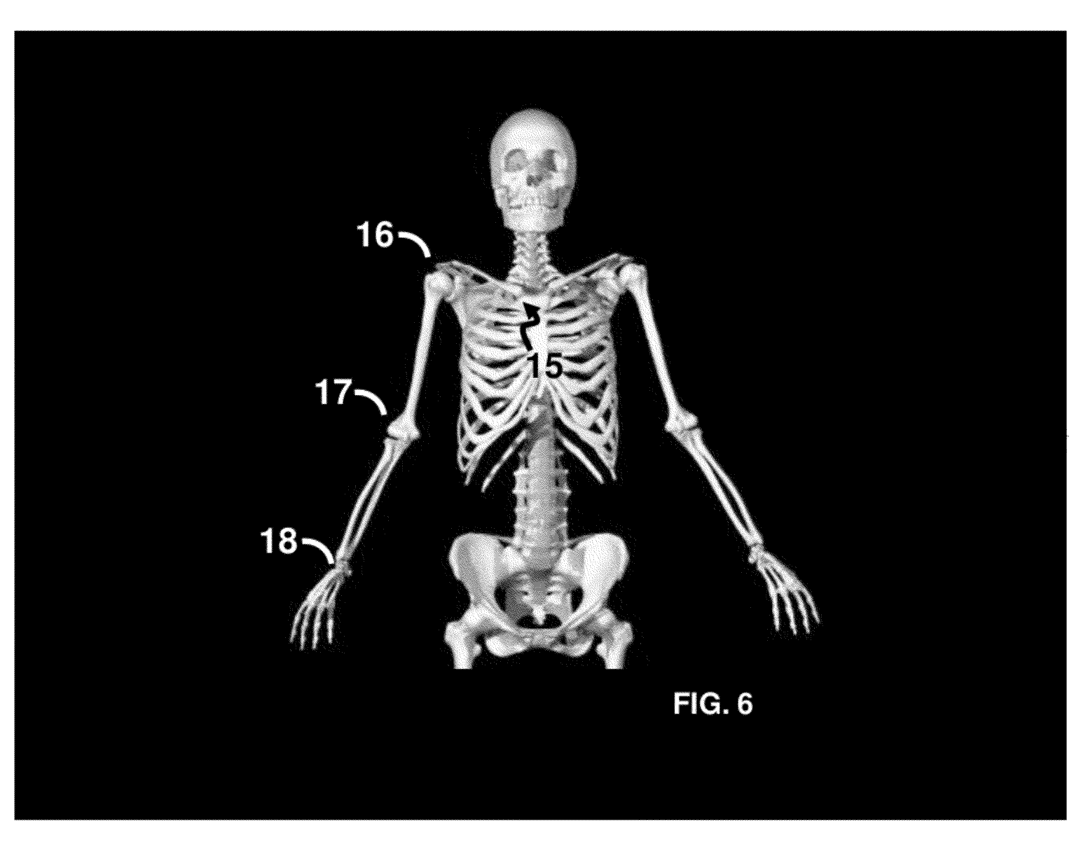

### DETAILED DESCRIPTION OF THE PREFERRED EMBODIMENTS

Embodiments of the present invention involve application of physical adjustments to a patient's body using manual application of force, or through manipulation with assistance of various mechanical equipment. In the treatment tables shown below, a directed application of force is applied in the manner and/or direction indicated for the listed body structures provided, and for example, P-A may indicate an application of force from the posterior to the anterior position of the named body structure. Some alternative embodiments of the present invention may utilize such instruments as an activator instrument, a toggle board, and a chiropractic table, all of which are described below.

A conventional activator instrument that may be used in accordance with the present invention is a prior-art type used in the chiropractic disciplines, and has features similar to a combination syringe and a pogo stick. The length of the activator used in embodiments of the present invention has a length of about 20 cm, although different sized activators may be used as the situation requires. The activator has a hard rubber foot with a diameter of about a centimeter along with an adjustable spring tensioner which presets the applied force. When pushed down, the activator delivers a small controlled mechanical “punch” to the specific area it is in contact with restoring motion to a restricted joint.

Another conventional instrument used in various embodiments of the present invention is what is known as a toggle board. The Thule toggle board is one particular type used in preferred embodiments herein. The Thule toggle board was originally designed for chiropractic treatment of the top vertebra in the cervical spine. It can also be used for the treatment of extremities for the correction of biomechanical joint dysfunctions. The toggle board comprises two sections, upper and lower. In one version used in aspects of the present invention, each section measures approximately 8 inches in length, 5.5 inches in width, and 1 inch thick. The upper section is connected to the lower section at one end by a 5 inch by 0.75 inch steel bracket which attaches to the outside borders of the lower section of the toggle board. The upper section is a half inch cushion surface over a half inch solid wood foundation. This section is upholstered with a vinyl-type material. The underside of the upper section has a 2.5 inch by 2 inch hard plastic square which is secured by four perimeter bolts. The lower portion of the toggle board is solid wood, such as oak. Five inches from the end of this board there is a 3 inch aluminum lever on the lateral surface of this board which when lifted raises a hard plastic peg located on the top side of this solid wood board. This peg lifts approximately 0.5 inches which presses against the opposing hard plastic 2.5 inch by 2 inch located on the underside of the vinyl upholstered piece. The raising of this lever arm raises the upper upholstered piece slightly over 0.5 inches at the opened end. On the opposite lateral side of the board there is a 0.75 inch diameter circular flat knob which can be turned to adjust the amount of tension on the peg, which allows this board to adapt to heavier or lighter extremity weight. If for example we are adjusting a joint within the foot, the foot is placed on the vinyl padded upper section of the board. The practitioner then applies a downward force, causing the hard plastic peg to release causing the upper section to drop on to the lower section. The momentum of the upper section falling with the extremity weight striking the stationary lower section causes a slight jarring of the joint, restoring the desired motion to the restricted joint.

A chiropractic table is used in various embodiments of the present invention. In a preferred embodiment, the chiropractic table is a conventional table such as the Hill Air Drop HA90C. The specifications for the preferred chiropractic table are as follows: electrically controlled height 21.5 to 30.5 inches; tilting headpiece - 30° negative and positive tilt; Air-Dual drop forward and straight-motion headpiece; Air-Thoracic breakaway; Air-Thoracic drop; Air-Lumbar drop; Air-Pelvic drop; Rocker foot pedal to raise or lower the table height; Air-powered foot control from foot end; Standard width—24 inches; Length—6 feet 3 inches; Foam top—2.5 inches; Arm rests, 13 inch face cut-out; and paper roll. The table is used with the patient either prone, supine or side lying, as specified herein. With various aspects of the present invention, adjustments are performed to areas of joint dysfunction in the extremities using the drop pieces mentioned above. In the preferred chiropractic table, these air drop pieces are supplied by a large air-storage tank and mini-compressor which are enclosed within the table's base skirting. A compressor runs periodically to replenish the air tank.

The preferred chiropractic table uses an Air-Breakaway controlled by a foot pedal. The pedal increases or decreases the air-spring pressure in the thoracic and lumbar sections providing a controlled recoil action. The table has electrically adjustable height. Height adjustment is actuated by a rocker foot pedal that is mounted to the base and can be accessed from either side of the table.

As noted with the toggle board, when the table piece drops there is a slight jarring of the joint, restoring the desired motion to the restricted joint. A directed manual “push” using mostly the patient's own body weight, is needed to activate the chiropractic table and toggle board drop piece mechanisms.

In alternative embodiments of the present invention, one diagnostic method used to determine the presence of joint dysfunction is called motion palpation. With motion palpation, the doctor sits behind the seated patient to examine this patient's spine. The doctor's left hand is commonly placed on the patient's left shoulder. The doctor's right hand is used by pressing with the flat of the first on the spinal segments, pushing forward slightly at each level. The doctor is checking for joint play (spring) between each vertebra. The normal actions of flexion, extension, left and right lateral flexion and rotation can be evaluated with this method. The joints of the pelvis, arms and legs also can be accurately motion palpated. Through motion palpation diagnosis, it can be determined at what segments joint dysfunction is present and when and where corrective adjustments are needed. Once treatment is administered, the affected area is re-palpated to see if normal joint function has been restored.

Also, as mentioned previously, through motion palpation techniques the practitioner may also determine which leg is the body-dominant leg by having the patient lie supine on the patient's back, with both legs initially straight. The practitioner alternatively brings each leg, one at a time to the patient's chest, and through motion palpation determines which leg requires more force to bend to the chest and/or has less range of motion, and that leg for purposes of the treatment will be established as the dominant leg. Those of skill in the relevant arts also recognize that other techniques may be used to determine the dominant leg. For most of the population, the right leg has found to be the dominant leg.

Although preferred embodiments of the present invention do not require direct manipulation of the cervical or thoracic spine, in various embodiments of the present invention, manual treatment may be applied to the cervical spine using conventional chiropractic Gonstead and Diversified methods. Manual treatment of the sacral and iliac regions of the patient's body uses the Diversified “side posture” (side lying) adjustment.

Treatment by Patterns of Adjustments in Sequentially-Ordered Steps

Embodiments of the present invention employ adjustment patterns applied in a structured manner in sequential, time-ordered steps. Once properly applied, the sequential adjustment patterns provoke an automatic corrective response in the patient and in most cases, the patient does not require future repetitive treatments. As mentioned above, some patients, however, may benefit from periodic treatments to the foot/ankle area as walking on hard, flat surfaces may cause dysfunction to subsequently arise. Embodiments may include applications of any predetermined number of adjustment patterns administered over any predetermined fixed or variable time period.

Any number of sequence steps may be utilized to apply adjustment patterns. In the preferred embodiment, 18 treatment steps are utilized in the treatment sequence, which is administered as a time-regulated primary cycle (steps 1-9 in a preferred embodiment) followed by a secondary cycle (steps 10-18 in a preferred embodiment). In any one treatment sequence step, one or more treatment patterns may be applied to the patient as desired to obtain a desired treatment goal. In any treatment pattern, one or more adjustments are applied to the indicated structure of the patient's body, and in a preferred embodiment, the adjustments are applied in a predetermined order. In the preferred embodiment, each of the 18 treatment steps is respectively administered in order once per day in sequential days (not necessarily consecutive calendar days), wherein all of the adjustments in the adjustment pattern specified for that treatment step are administered in a predetermined order that day. In another embodiment, the 18-step treatment sequence may be shortened by combining one or more treatment patterns on any treatment day. The sequence steps may be spaced over any desired time period, such as daily, weekly or monthly according to patient preference and treatment results. In a preferred embodiment of the present invention called the GAITLINK method, one adjustment pattern comprising one or more sequential adjustments are applied per sequence step, with the sequence steps occurring once per day over a period of eighteen days (not necessarily consecutive days). Preferably, the patient rests at least overnight between applications of each treatment step. Referring to tables 1-18 below, in the preferred embodiment, the twenty-three adjustments shown in the adjustment pattern for Table (Sequence Step) 1 would be administered in order on day 1 of the treatment, then the ten adjustments of the adjustment pattern shown in Table (Sequence Step) 2 would be applied in the order shown on day 2, the three adjustments shown in the adjustment pattern for Table (Sequence Step) 3 would be administered in order on day 3 of the treatment, and so on until the 18-step sequence is completed. In the tables below, the primary treatment cycle comprises sequence steps 1-9 and the secondary treatment cycle comprises steps 10-18.

Alternatively, the eighteen-step treatment sequence could comprise the administration of multiple treatment patterns per treatment step, in any order to achieve a desired treatment goal. For example, the eighteen-step treatment sequence could be shortened by one step by combining any two treatment steps on a particular day. In one preferred alternate embodiment, both adjustment patterns of Sequence Step 9 (Table 9) and Sequence Step 10 (Table 10) are applied on the same treatment day, shortening the total treatment time by one day. In yet another embodiment shown in tables 1 A-20A, the process could be lengthened by breaking up one or more treatment patterns onto different days, and in the embodiment shown, the proximal clavicle is now treated on a day separate from the sacrum/ilium to accomplish desired treatment goals such as locking or unlocking pelvic structures prior to treatment beginning for the upper body. Those of skill in the art also understand that sequence steps may be repeated as desired, and additional or different adjustment patterns may be utilized to obtain a desired treatment goal.

In the tables below, abbreviations used in the “Preferred Instrument” column are as follows: the activator instrument will be shown as (AT); the toggle board will be shown as (TT); the chiropractic table will be shown as (CT) and manual treatment will be shown as (MA). Patient Position abbreviations are as follows: Supine (SU); Prone (PR); Side lying (SL); Seated in chair (CH); and Standing (ST). The “#” column corresponds to the order of application of each adjustment to the patient.

TABLES 1-18: Eighteen-Step Treatment Sequence

TABLE 1
Sequence Step 1

| | | | Pa- | Pre- |
|---|---|---|---|---|
| | | | tient | ferred |
| Structure | | | Posi- | Instru- |
| Treated | # | Adjustment Pattern Element | tion | ment |
| Foot of | 1 | Ankle Mortise Long Axis Extension | SU | MA |
| Dominant | 2 | First Ray Long Axis Extension | SU | MA |
| Leg (for | 3 | Subtalar Joint Long Axis Extension | SU | MA |
| example, | 4 | Subtalar Joint Medial to Lateral Glide | SU | TT |
| patient's | 5 | Subtalar Joint Lateral to Medial Glide | SL | TT |
| right side) | 6 | Subtalar Joint Medial to Lateral Tilt | SU | TT |
| | 7 | Subtalar Joint Lateral to Medial Tilt | SL | TT |
| | 8 | Talar Tilt Medial to Lateral | SU | TT |
| | 9 | Talar Tilt Lateral to Medial | SL | TT |
| | 10 | P-A Shear Calcaneus | PR | TT |
| | 11 | Transtarsal Joint Force Application | SU | TT |
| | 12 | Ankle Mortise A-P Shear with Internal | SU | MA |
| | | Rotation Tibia | | |
| | 13 | Ankle Mortise P-A Shear with External | SU | TT |
| | | Rotation Tibia | | |
| | 14 | A-P Calcaneus on Talus | SU | TT |
| | 15 | Calcaneocuboid Dorsal to Plantar Spin | SU | TT |
| | 16 | Calcaneocuboid Plantar to Dorsal Spin | SU | TT |
| | 17 | Calcaneocuboid Lateral to Medial Glide | SU | TT |
| | 18 | TCN Joint Dorsal to Plantar Spin | SU | TT |
| | 19 | First Cuneonavicular Joint Dorsal/ | SU | TT |
| | | Plantar Spin | | |
| | 20 | First Ray Complex Dorsal/Plantar Spin | SU | TT |
| | 21 | TCN Joint Plantar to Dorsal Spin | SU | TT |
| | 22 | First Cumeonavicular Joint | SU | TT |
| | | Plantar/Dorsal Spin | | |
| | 23 | First Ray Complex Plantar/Dorsal Spin | SU | TT |

TABLE 2
Sequence Step. 2

| Structure | | | Patient | Preferred |
|---|---|---|---|---|
| Treated | # | Adjustment Pattern Element | Position | Instrument |
| Knee of | 1 | A-P Tibia on Femur | SU | CT |
| Dominant | 2 | Flexion/Internal Rotation Tibia | SU | TT |
| Leg (for | | on Femur | | |
| example, | 3 | Extension/External Rotation Tibia | SU | TT |
| patient's | | on Femur | | |
| right side) | 4 | Internal to External Fibular | SU | TT |
| | | Rotation on Tibia (P-A) | | |
| | 5 | External to Internal Fibular | SU | AT |
| | | Rotation on Tibia (A-P) | | |
| | 6 | P-A Proximal Tibia on Distal | SU | TT |
| | | Femur | | |
| | 7 | Lateral to Medial Femur on Tibia | SU | TT |
| | 8 | Medial to Lateral Tibia on Femur | SU | TT |
| | 9 | Medial to Lateral Femur on Tibia | SU | TT |
| | 10 | Lateral to Medial Tibia on Femur | SU | TT |

TABLE 3
Sequence Step 3

| Structure | | | Patient | Preferred |
|---|---|---|---|---|
| Treated | # | Adjustment Pattern Element | Position | Instrument |
| Hip | 1 | Circumduction Femur in | SU | CT |
| proximate | | Acetabulum | | |
| Dominant | 2 | Superior to Inferior Distraction | SU | MA |
| Leg (for | | Femur | | |
| example, | 3 | Gluteus Maximus/Piriformis 1B | SU | CT |
| patient's | | Afferent Stretch | | |
| right side) | | | | |

TABLE 4
Sequence Step 4

| Structure | | | Patient | Preferred |
|---|---|---|---|---|
| Treated | # | Adjustment Pattern Element | Position | Instrument |
| Pubic | 1 | Right Pubic Ramus Superior to | SU | CT |
| Symphysis | | Inferior | | |
| proximate | 2 | Adduction with resistance at | SU | MA |
| Dominant | | Symphysis | | |
| Leg (for | | | | |
| example, | | | | |
| patient's | | | | |
| right side) | | | | |

TABLE 5
Sequence Step 5

| Structure | | | Patient | Preferred |
|---|---|---|---|---|
| Treated | # | Adjustment Pattern Element | Position | Instrument |
| Lower | 1 | P-A Sacrum on Ilium (involved | SL | CT |
| Sacrum | | side down) | | |
| proximate | | | | |
| Dominant | | | | |
| Leg (for | | | | |
| example, | | | | |
| patient's | | | | |
| right side) | | | | |
| Upper Ilium | 2 | P-A I-S Ilium on Sacrum | SL | CT |
| proximate | | | | |
| Dominant | | | | |
| Leg (for | | | | |
| example, | | | | |
| patient's | | | | |
| right side) | | | | |

TABLE 6
Sequence Step 6

| Structure | | | Patient | Preferred |
|---|---|---|---|---|
| Treated | # | Adjustment Pattern Element | Position | Instrument |
| Upper | 1 | P-A Sacrum on Ilium (involved | SL | CT |
| Sacrum | | side down) | | |
| proximate | | | | |
| Non- | | | | |
| dominant | | | | |
| Leg (for | | | | |
| example, | | | | |
| patient's | | | | |
| left side) | | | | |
| Upper Ilium | 2 | P-A S-I Ilium on Sacrum | SL | CT |
| proximate | | | | |
| Non- | | | | |
| dominant | | | | |
| Leg (for | | | | |
| example, | | | | |
| patient's | | | | |
| left side) | | | | |
| Proximal | 1 | P-A M-L Proximal Clavicle | SU | CT |
| Clavicle on | | force application | | |
| Non- | | | | |
| dominant | | | | |
| side (for | | | | |
| example, | | | | |
| patient's | | | | |
| left side) | | | | |

TABLE 7
Sequence Step 7

| Structure | | | Patient | Preferred |
|---|---|---|---|---|
| Treated | # | Adjustment Pattern Element | Position | Instrument |
| Shoulder | 1 | Circumduction humerus in | SU | CT |
| on Non- | | Glenoid fossa lateral/medial/A-P | | |
| dominant | 2 | Internal rotation humerus in | SU | CT |
| side (for | | Gleniod fossa medial/lateral/A-P | | |
| example, | 3 | External rotation humerus in | SU | CT |
| patient's | | Glenoid fossa medial/lateral/A-P | | |
| left side) | 4 | P-A S-I distal clavicle on | CH | AT |
| | | Acromion process w/ internal | | |
| | | rotation | | |
| | 5 | Internal scapular glide on | CH | MA |
| | | abduction/ humerus/Glenoid fossa | | |

TABLE 8
Sequence Step 8

| Structure | | | Patient | Preferred |
|---|---|---|---|---|
| Treated | # | Adjustment Pattern Element | Position | Instrument |
| Elbow on | 1 | External to internal rotation of | ST | MA |
| Non- | | proximal radius in ulnar notch | | |
| dominant | 2 | P-A proximal ulna on distal | ST | MA |
| side (for | | humerus | | |
| example, | 3 | Medial to lateral-lateral to medial | ST | MA |
| patient's | | glide of proximal ulna on distal | | |
| left side) | | humerus | | |

TABLE 9
Sequence Step 9

| Structure | | | Patient | Preferred |
|---|---|---|---|---|
| Treated | # | Adjustment Pattern Element | Position | Instrument |
| Wrist on | 1 | P-A Distal Radius on Proximal | ST | MA |
| Non- | | Carpals | | |
| dominant | 2 | P-A Distal Ulna on Fibrocartiledge | ST | MA |
| side (for | 3 | P-A Distal Radioulnar Joint | ST | MA |
| example, | 4 | P-A A-P Glide Carpals | ST | MA |
| patient's | 5 | Superior to Inferior First | ST | MA |
| left side) | | Metacarpal on Trapezium | | |

TABLE 10
Sequence Step 10

| Structure | | | Patient | Preferred |
|---|---|---|---|---|
| Treated | # | Adjustment Pattern Element | Position | Instrument |
| Foot of | 1 | Ankle Mortise Long Axis | SU | MA |
| Non- | | Extension | | |
| dominant | 2 | First Ray Long Axis Extension | SU | MA |
| Leg (for | 3 | Subtalar Joint Long Axis Extension | SU | MA |
| example, | 4 | Subtalar Joint Medial to Lateral | SU | TT |
| patient's | | Glide | | |
| left side) | 5 | Subtalar Joint Lateral to Medial | SL | TT |
| | | Glide | | |
| | 6 | Subtalar Joint Medial to Lateral | SU | TT |
| | | Tilt | | |
| | 7 | Subtalar Joint Lateral to Medial | SL | TT |
| | | Tilt | | |
| | 8 | Talar Tilt Medial to Lateral | SU | TT |
| | 9 | Talar Tilt Lateral to Medial | SL | TT |
| | 10 | P-A Shear Calcaneus | PR | TT |
| | 11 | Transtarsal Joint Force Application | SU | TT |
| | 12 | Ankle Mortise A-P Shear with | SU | MA |
| | | Internal Rotation Tibia | | |
| | 13 | Ankle Mortise P-A Shear with | SU | TT |
| | | External Rotation Tibia | | |
| | 14 | A-P Calcaneus on Talus | SU | TT |
| | 15 | Calcaneocuboid Dorsal to Plantar | SU | TT |
| | | Spin | | |
| | 16 | Calcaneocuboid Plantar to Dorsal | SU | TT |
| | | Spin | | |
| | 17 | Calcaneocuboid Lateral to Medial | SU | TT |
| | | Glide | | |
| | 18 | TCN Joint Dorsal to Plantar Spin | SU | TT |
| | 19 | First Cuneonavicular Joint Dorsal/ | SU | TT |
| | | Plantar Spin | | |
| | 20 | First Ray Complex Dorsal/ | SU | TT |
| | | Plantar Spin | | |
| | 21 | TCN Joint Plantar to Dorsal Spin | SU | TT |
| | 22 | First Cumeonavicular Joint | SU | TT |
| | | Plantar/Dorsal Spin | | |
| | 23 | First Ray Complex Plantar/ | SU | TT |
| | | Dorsal Spin | | |

TABLE 11
Sequence Step. 11

| Structure | | | Patient | Preferred |
|---|---|---|---|---|
| Treated | # | Adjustment Pattern Element | Position | Instrument |
| Knee of | 1 | A-P Tibia on Femur | SU | CT |
| Non- | 2 | Flexion/Internal Rotation Tibia | SU | TT |
| dominant | | on Femur | | |
| Leg (for | 3 | Extension/External Rotation Tibia | SU | TT |
| example, | | on Femur | | |
| patient's | 4 | Internal to External Fibular | SU | TT |
| left side) | | Rotation on Tibia (P-A) | | |
| | 5 | External to Internal Fibular | SU | AT |
| | | Rotation on Tibia (A-P) | | |
| | 6 | P-A Proximal Tibia on Distal | SU | TT |
| | | Femur | | |
| | 7 | Lateral to Medial Femur on Tibia | SU | TT |
| | 8 | Medial to Lateral Tibia on Femur | SU | TT |
| | 9 | Medial to Lateral Femur on Tibia | SU | TT |
| | 10 | Lateral to Medial Tibia on Femur | SU | TT |

TABLE 12
Sequence Step 12

| Structure | | | Patient | Preferred |
|---|---|---|---|---|
| Treated | # | Adjustment Pattern Element | Position | Instrument |
| Hip | 1 | Circumduction Femur in | SU | CT |
| proximate | | Acetabulum | | |
| Non- | 2 | Superior to Inferior Distraction | SU | MA |
| dominant | | Femur | | |
| Leg (for | 3 | Gluteus Maximus/Piriformis 1B | SU | CT |
| example, | | Afferent Stretch | | |
| patient's | | | | |
| left side) | | | | |

TABLE 13
Sequence Step 13

| Structure | | | Patient | Preferred |
|---|---|---|---|---|
| Treated | # | Adjustment Pattern Element | Position | Instrument |
| Pubic | 1 | Right Pubic Ramus Superior to | SU | CT |
| Symphysis | | Inferior | | |
| proximate | 2 | Adduction with resistance at | SU | MA |
| Non- | | Symphysis | | |
| dominant | | | | |
| Leg (for | | | | |
| example, | | | | |
| patient's | | | | |
| left side) | | | | |

TABLE 14
Sequence Step 14

| Structure | | | Patient | Preferred |
|---|---|---|---|---|
| Treated | # | Adjustment Pattern Element | Position | Instrument |
| Lower | 1 | P-A Sacrum on Ilium (involved | SL | CT |
| Sacrum | | side down) | | |
| proximate | | | | |
| Non- | | | | |
| dominant | | | | |
| Leg (for | | | | |
| example, | | | | |
| patient's | | | | |
| left side) | | | | |
| Upper Ilium | 2 | P-A I-S Ilium on Sacrum | SL | CT |
| proximate | | | | |
| Non- | | | | |
| dominant | | | | |
| Leg (for | | | | |
| example, | | | | |
| patient's | | | | |
| left side) | | | | |

TABLE 15
Sequence Step 15

| Structure | | | Patient | Preferred |
|---|---|---|---|---|
| Treated | # | Adjustment Pattern Element | Position | Instrument |
| Upper | 1 | P-A Sacrum on Ilium (involved | SL | CT |
| Sacrum | | side down) | | |
| proximate | | | | |
| Dominant | | | | |
| Leg (for | | | | |
| example, | | | | |
| patient's | | | | |
| right side) | | | | |
| Upper Ilium | 2 | P-A S-I Ilium on Sacrum | SL | CT |
| proximate | | | | |
| Dominant | | | | |
| Leg (for | | | | |
| example, | | | | |
| patient's | | | | |
| right side) | | | | |
| Proximal | 1 | P-A M-L Proximal Clavicle | SU | CT |
| Clavicle on | | force application | | |
| Dominant | | | | |
| side (for | | | | |
| example, | | | | |
| patient's | | | | |
| right side) | | | | |

TABLE 16
Sequence Step 16

| Structure | | | Patient | Preferred |
|---|---|---|---|---|
| Treated | # | Adjustment Pattern Element | Position | Instrument |
| Shoulder | 1 | Circumduction humerus in Glenoid | SU | CT |
| on | | fossa lateral/medial/A-P | | |
| Dominant | 2 | Internal rotation humerus in | SU | CT |
| side (for | | Gleniod fossa medial/lateral/A-P | | |
| example, | 3 | External rotation humerus in | SU | CT |
| patient's | | Glenoid fossa medial/lateral/A-P | | |
| right side) | 4 | P-A S-I distal clavicle on | CH | AT |
| | | Acromion process w/ internal | | |
| | | rotation | | |
| | 5 | Internal scapular glide on | CH | MA |
| | | abduction/humerus/Glenoid fossa | | |

TABLE 17
Sequence Step 17

| Structure | | | Patient | Preferred |
|---|---|---|---|---|
| Treated | # | Adjustment Pattern Element | Position | Instrument |
| Elbow on | 1 | External to internal rotation of | ST | MA |
| Dominant | | proximal radius in ulnar notch | | |
| side (for | 2 | P-A proximal ulna on distal | ST | MA |
| example, | | humerus | | |
| patient's | 3 | Medial to lateral - lateral to medial | ST | MA |
| right side) | | glide of proximal ulna on distal | | |
| | | humerus | | |

TABLE 18
Sequence Step 18

| Structure | | | Patient | Preferred |
|---|---|---|---|---|
| Treated | # | Adjustment Pattern Element | Position | Instrument |
| Wrist on | 1 | P-A Distal Radius on Proximal | ST | MA |
| Dominant | | Carpals | | |
| side (for | 2 | P-A Distal Ulna on Fibrocartiledge | ST | MA |
| example, | 3 | P-A Distal Radioulnar Joint | ST | MA |
| patient's | 4 | P-A A-P Glide Carpals | ST | MA |
| right side) | 5 | Superior to Inferior First | ST | MA |
| | | Metacarpal on Trapezium | | |

### Tables 1A-20A: Twenty-Step Treatment Sequence

TABLE 1A
Sequence Step 1

| Structure | | | Patient | Preferred |
|---|---|---|---|---|
| Treated | # | Adjustment Pattern Element | Position | Instrument |
| Foot of | 1 | Ankle Mortise Long Axis | SU | MA |
| Dominant | | Extension | | |
| Leg (for | 2 | First Ray Long Axis Extension | SU | MA |
| example, | 3 | Subtalar Joint Long Axis Extension | SU | MA |
| patient's | 4 | Subtalar Joint Medial to Lateral | SU | TT |
| right side) | | Glide | | |
| | 5 | Subtalar Joint Lateral to Medial | SL | TT |
| | | Glide | | |
| | 6 | Subtalar Joint Medial to Lateral | SU | TT |
| | | Tilt | | |
| | 7 | Subtalar Joint Lateral to Medial | SL | TT |
| | | Tilt | | |
| | 8 | Talar Tilt Medial to Lateral | SU | TT |
| | 9 | Talar Tilt Lateral to Medial | SL | TT |
| | 10 | P-A Shear Calcaneus | PR | TT |
| | 11 | Transtarsal Joint Force Application | SU | TT |
| | 12 | Ankle Mortise A-P Shear with | SU | MA |
| | | Internal Rotation Tibia | | |
| | 13 | Ankle Mortise P-A Shear with | SU | TT |
| | | External Rotation Tibia | | |
| | 14 | A-P Calcaneus on Talus | SU | TT |
| | 15 | Calcaneocuboid Dorsal to Plantar | SU | TT |
| | | Spin | | |
| | 16 | Calcaneocuboid Plantar to Dorsal | SU | TT |
| | | Spin | | |
| | 17 | Calcaneocuboid Lateral to Medial | SU | TT |
| | | Glide | | |
| | 18 | TCN Joint Dorsal to Plantar Spin | SU | TT |
| | 19 | First Cuneonavicular Joint Dorsal/ | SU | TT |
| | | Plantar Spin | | |
| | 20 | First Ray Complex Dorsal/ | SU | TT |
| | | Plantar Spin | | |
| | 21 | TCN Joint Plantar to Dorsal Spin | SU | TT |
| | 22 | First Cumeonavicular Joint | SU | TT |
| | | Plantar/Dorsal Spin | | |
| | 23 | First Ray Complex Plantar/ | SU | TT |
| | | Dorsal Spin | | |

TABLE 2A
Sequence Step. 2

| Structure | | | Patient | Preferred |
|---|---|---|---|---|
| Treated | # | Adjustment Pattern Element | Position | Instrument |
| Knee of | 1 | A-P Tibia on Femur | SU | CT |
| Dominant | 2 | Flexion/Internal Rotation Tibia | SU | TT |
| Leg (for | | on Femur | | |
| example, | 3 | Extension/External Rotation Tibia | SU | TT |
| patient's | | on Femur | | |
| right side) | 4 | Internal to External Fibular | SU | TT |
| | | Rotation on Tibia (P-A) | | |
| | 5 | External to Internal Fibular | SU | AT |
| | | Rotation on Tibia (A-P) | | |
| | 6 | P-A Proximal Tibia on Distal | SU | TT |
| | | Femur | | |
| | 7 | Lateral to Medial Femur on Tibia | SU | TT |
| | 8 | Medial to Lateral Tibia on Femur | SU | TT |
| | 9 | Medial to Lateral Femur on Tibia | SU | TT |
| | 10 | Lateral to Medial Tibia on Femur | SU | TT |

TABLE 3A
Sequence Step 3

| Structure | | | Patient | Preferred |
|---|---|---|---|---|
| Treated | # | Adjustment Pattern Element | Position | Instrument |
| Hip | 1 | Circumduction Femur in | SU | CT |
| proximate | | Acetabulum | | |
| Dominant | 2 | Superior to Inferior Distraction | SU | MA |
| Leg (for | | Femur | | |
| example, | 3 | Gluteus Maximus/Piriformis 1B | SU | CT |
| patient's | | Afferent Stretch | | |
| right side) | | | | |

TABLE 4A
Sequence Step 4

| Structure | | | Patient | Preferred |
|---|---|---|---|---|
| Treated | # | Adjustment Pattern Element | Position | Instrument |
| Pubic | 1 | Right Pubic Ramus Superior to | SU | CT |
| Symphysis | | Inferior | | |
| proximate | 2 | Adduction with resistance at | SU | MA |
| Dominant | | Symphysis | | |
| Leg (for | | | | |
| example, | | | | |
| patient's | | | | |
| right side) | | | | |

TABLE 5A
Sequence Step 5

| Structure | | | Patient | Preferred |
|---|---|---|---|---|
| Treated | # | Adjustment Pattern Element | Position | Instrument |
| Lower | 1 | P-A Sacrum on Ilium (involved | SL | CT |
| Sacrum | | side down) | | |
| proximate | | | | |
| Dominant | | | | |
| Leg (for | | | | |
| example, | | | | |
| patient's | | | | |
| right side) | | | | |
| Upper Ilium | 2 | P-A I-S Ilium on Sacrum | SL | CT |
| proximate | | | | |
| Dominant | | | | |
| Leg (for | | | | |
| example, | | | | |
| patient's | | | | |
| right side) | | | | |

TABLE 6A
Sequence Step 6

| Structure | | | Patient | Preferred |
|---|---|---|---|---|
| Treated | # | Adjustment Pattern Element | Position | Instrument |
| Upper | 1 | P-A Sacrum on Ilium (involved | SL | CT |
| Sacrum | | side down) | | |
| proximate | | | | |
| Non- | | | | |
| dominant | | | | |
| Leg (for | | | | |
| example, | | | | |
| patient's | | | | |
| left side) | | | | |
| Upper Ilium | 2 | P-A S-I Ilium on Sacrum | SL | CT |
| proximate | | | | |
| Non- | | | | |
| dominant | | | | |
| Leg (for | | | | |
| example, | | | | |
| patient's | | | | |
| left side) | | | | |

TABLE 7A
Sequence Step 7

| Structure | | | Patient | Preferred |
|---|---|---|---|---|
| Treated | # | Adjustment Pattern Element | Position | Instrument |
| Proximal | 1 | P-A M-L Proximal Clavicle force | SU | CT |
| Clavicle | | application | | |
| on Non- | | | | |
| dominant | | | | |
| side (for | | | | |
| example, | | | | |
| patient's | | | | |
| left side) | | | | |

TABLE 8A
Sequence Step 8

| Structure | | | Patient | Preferred |
|---|---|---|---|---|
| Treated | # | Adjustment Pattern Element | Position | Instrument |
| Shoulder | 1 | Circumduction humerus in Glenoid | SU | CT |
| on Non- | | fossa lateral/medial/A-P | | |
| dominant | 2 | Internal rotation humerus in | SU | CT |
| side (for | | Gleniod fossa medial/lateral/ | | |
| example, | | A-P | | |
| patient's | 3 | External rotation humerus in | SU | CT |
| left side) | | Glenoid fossa medial/lateral/ | | |
| | | A-P | | |
| | 4 | P-A S-I distal clavicle on | CH | AT |
| | | Acromion process w/ internal | | |
| | | rotation | | |
| | 5 | Internal scapular glide on | CH | MA |
| | | abduction/humerus/Glenoid | | |
| | | fossa | | |

TABLE 9A
Sequence Step 9

| Structure | | | Patient | Preferred |
|---|---|---|---|---|
| Treated | # | Adjustment Pattern Element | Position | Instrument |
| Elbow on | 1 | External to internal rotation of | ST | MA |
| Non- | | proximal radius in ulnar | | |
| dominant | | notch | | |
| side (for | 2 | P-A proximal ulna on distal | ST | MA |
| example, | | humerus | | |
| patient's | 3 | Medial to lateral - lateral to medial | ST | MA |
| left side) | | glide of proximal ulna on distal | | |
| | | humerus | | |

TABLE 10A
Sequence Step 10

| Structure | | | Patient | Preferred |
|---|---|---|---|---|
| Treated | # | Adjustment Pattern Element | Position | Instrument |
| Wrist on | 1 | P-A Distal Radius on Proximal | ST | MA |
| Non- | | Carpals | | |
| dominant | 2 | P-A Distal Ulna on Fibrocartiledge | ST | MA |
| side (for | 3 | P-A Distal Radioulnar Joint | ST | MA |
| example, | 4 | P-A A-P Glide Carpals | ST | MA |
| patient's | 5 | Superior to Inferior First | ST | MA |
| left side) | | Metacarpal on Trapezium | | |

TABLE 11A
Sequence Step 11

| Structure | | | Patient | Preferred |
|---|---|---|---|---|
| Treated | # | Adjustment Pattern Element | Position | Instrument |
| Foot of | 1 | Ankle Mortise Long Axis Extension | SU | MA |
| Non- | 2 | First Ray Long Axis Extension | SU | MA |
| dominant | 3 | Subtalar Joint Long Axis Extension | SU | MA |
| Leg (for | 4 | Subtalar Joint Medial to Lateral Glide | SU | TT |
| example, | 5 | Subtalar Joint Lateral to Medial Glide | SL | TT |
| patient's | 6 | Subtalar Joint Medial to Lateral Tilt | SU | TT |
| left side) | 7 | Subtalar Joint Lateral to Medial Tilt | SL | TT |
| | 8 | Talar Tilt Medial to Lateral | SU | TT |
| | 9 | Talar Tilt Lateral to Medial | SL | TT |
| | 10 | P-A Shear Calcaneus | PR | TT |
| | 11 | Transtarsal Joint Force Application | SU | TT |
| | 12 | Ankle Mortise A-P Shear with Internal | SU | MA |
| | | Rotation Tibia | | |
| | 13 | Ankle Mortise P-A Shear with External | SU | TT |
| | | Rotation Tibia | | |
| | 14 | A-P Calcaneus on Talus | SU | TT |
| | 15 | Calcaneocuboid Dorsal to Plantar Spin | SU | TT |
| | 16 | Calcaneocuboid Plantar to Dorsal Spin | SU | TT |
| | 17 | Calcaneocuboid Lateral to Medial Glide | SU | TT |
| | 18 | TCN Joint Dorsal to Plantar Spin | SU | TT |
| | 19 | First Cuneonavicular Joint Dorsal/Plantar Spin | SU | TT |
| | 20 | First Ray Complex Dorsal/Plantar Spin | SU | TT |
| | 21 | TCN Joint Plantar to Dorsal Spin | SU | TT |
| | 22 | First Cumeonavicular Joint | SU | TT |
| | | Plantar/Dorsal Spin | | |
| | 23 | First Ray Complex Plantar/Dorsal Spin | SU | TT |

TABLE 12A
Sequence Step. 12

| Structure | | | Patient | Preferred |
|---|---|---|---|---|
| Treated | # | Adjustment Pattern Element | Position | Instrument |
| Knee of | 1 | A-P Tibia on Femur | SU | CT |
| Non- | 2 | Flexion/Internal Rotation Tibia | SU | TT |
| dominant | | on Femur | | |
| Leg (for | 3 | Extension/External Rotation | SU | TT |
| example, | | Tibia on Femur | | |
| patient's | 4 | Internal to External Fibular | SU | TT |
| left side) | | Rotation on Tibia (P-A) | | |
| | 5 | External to Internal Fibular | SU | AT |
| | | Rotation on Tibia (A-P) | | |
| | 6 | P-A Proximal Tibia on Distal | SU | TT |
| | | Femur | | |
| | 7 | Lateral to Medial Femur on Tibia | SU | TT |
| | 8 | Medial to Lateral Tibia on Femur | SU | TT |
| | 9 | Medial to Lateral Femur on Tibia | SU | TT |
| | 10 | Lateral to Medial Tibia on Femur | SU | TT |

TABLE 13A
Sequence Step 13

| Structure | | | Patient | Preferred |
|---|---|---|---|---|
| Treated | # | Adjustment Pattern Element | Position | Instrument |
| Hip | 1 | Circumduction Femur in | SU | CT |
| proximate | | Acetabulum | | |
| Non- | 2 | Superior to Inferior Distraction | SU | MA |
| dominant | | Femur | | |
| Leg (for | 3 | Gluteus Maximus/Piriformis 1B | SU | CT |
| example, | | Afferent Stretch | | |
| patient's | | | | |
| left side) | | | | |

TABLE 14A
Sequence Step 14

| Structure | | | Patient | Preferred |
|---|---|---|---|---|
| Treated | # | Adjustment Pattern Element | Position | Instrument |
| Pubic | 1 | Right Pubic Ramus Superior to | SU | CT |
| Symphysis | | Inferior | | |
| proximate | 2 | Adduction with resistance at | SU | MA |
| Non- | | Symphysis | | |
| dominant | | | | |
| Leg (for | | | | |
| example, | | | | |
| patient's | | | | |
| left side) | | | | |

TABLE 15A
Sequence Step 15

| Structure | | | Patient | Preferred |
|---|---|---|---|---|
| Treated | # | Adjustment Pattern Element | Position | Instrument |
| Lower | 1 | P-A Sacrum on Ilium (involved | SL | CT |
| Sacrum | | side down) | | |
| proximate | | | | |
| Non- | | | | |
| dominant | | | | |
| Leg (for | | | | |
| example, | | | | |
| patient's | | | | |
| left side) | | | | |
| Upper Ilium | 2 | P-A I-S Ilium on Sacrum | SL | CT |
| proximate | | | | |
| Non- | | | | |
| dominant | | | | |
| Leg (for | | | | |
| example, | | | | |
| patient's | | | | |
| left side) | | | | |

TABLE 16A
Sequence Step 16

| Structure | | | Patient | Preferred |
|---|---|---|---|---|
| Treated | # | Adjustment Pattern Element | Position | Instrument |
| Upper | 1 | P-A Sacrum on Ilium (involved | SL | CT |
| Sacrum | | side down) | | |
| proximate | | | | |
| Dominant | | | | |
| Leg (for | | | | |
| example, | | | | |
| patient's | | | | |
| right side) | | | | |
| Upper Ilium | 2 | P-A S-I Ilium on Sacrum | SL | CT |
| proximate | | | | |
| Dominant | | | | |
| Leg (for | | | | |
| example, | | | | |
| patient's | | | | |
| right side) | | | | |

TABLE 17A
Sequence Step 17

| Structure | | | Patient | Preferred |
|---|---|---|---|---|
| Treated | # | Adjustment Pattern Element | Position | Instrument |
| Proximal | 1 | P-A M-L Proximal Clavicle force | SU | CT |
| Clavicle | | application | | |
| on | | | | |
| Dominant | | | | |
| side (for | | | | |
| example, | | | | |
| patient's | | | | |
| right side) | | | | |

TABLE 18A
Sequence Step 18

| Structure | | | Patient | Preferred |
|---|---|---|---|---|
| Treated | # | Adjustment Pattern Element | Position | Instrument |
| Shoulder | 1 | Circumduction humerus in | SU | CT |
| on | | Glenoid fossa lateral/medial/A-P | | |
| Dominant | 2 | Internal rotation humerus in | SU | CT |
| side (for | | Gleniod fossa medial/lateral/A-P | | |
| example, | 3 | External rotation humerus in | SU | CT |
| patient's | | Glenoid fossa medial/lateral/A-P | | |
| right side) | 4 | P-A S-I distal clavicle on | CH | AT |
| | | Acromion process w/ internal | | |
| | | rotation | | |
| | 5 | Internal scapular glide on | CH | MA |
| | | abduction/humerus/Glenoid | | |
| | | fossa | | |

TABLE 19A
Sequence Step 19

| Structure | | | Patient | Preferred |
|---|---|---|---|---|
| Treated | # | Adjustment Pattern Element | Position | Instrument |
| Elbow on | 1 | External to internal rotation of | ST | MA |
| Dominant | | proximal radius in ulnar notch | | |
| side (for | 2 | P-A proximal ulna on distal | ST | MA |
| example, | | humerus | | |
| patient's | 3 | Medial to lateral - lateral to medial | ST | MA |
| right side) | | glide of proximal ulna on | | |
| | | distal humerus | | |

TABLE 20A
Sequence Step 20

| Structure | | | Patient | Preferred |
|---|---|---|---|---|
| Treated | # | Adjustment Pattern Element | Position | Instrument |
| Wrist on | 1 | P-A Distal Radius on Proximal | ST | MA |
| Dominant | | Carpals | | |
| side (for | 2 | P-A Distal Ulna on Fibrocartiledge | ST | MA |
| example, | 3 | P-A Distal Radioulnar Joint | ST | MA |
| patient's | 4 | P-A A-P Glide Carpals | ST | MA |
| right side) | 5 | Superior to Inferior First | ST | MA |
| | | Metacarpal on Trapezium | | |

### Logan Basic Technique-Style Modified Embodiment

An additional embodiment provides for treatment of the sacrotuberous ligament and/or long dorsal ligament as replacement for or as an adjunct to various hip and/or pelvic structure treatments mentioned previously. The sacrotuberous ligament is can be found at the lower and back part of the pelvis.

In general, the Logan Basic Technique is a prior art chiropractic technique wherein with the patient lies face down (prone), and the practitioner places a very light pressure on a pre-determined “leverage spot” on the patient's body; for instance, the sacral bone in the low back. In an embodiment of the present invention, the practitioner uses the Logan Basic Technique to apply a very light force to the sacrotuberous ligament and/or long dorsal ligament as and adjunct to or replacement of various pelvic treatments. In one exemplary modification of the preferred 18-Step treatment sequence shown in Tables 1-18, in sequence step 5, treatment of the dominant-side lower sacrum and upper ilium would be replaced by application of Logan-Basic Technique pressure by application by the practitioner's finger to the patient's dominant-side sacrotuberous ligament (proximal the patient's dominant side sacrum). Then in sequence step 6, treatment of the non-dominant-side upper sacrum and upper ilium would be replaced by application of Logan-Basic Technique pressure by the practitioner's finger to the patient's non-dominant-side sacrotuberous ligament and/or long dorsal ligament (treatment to the proximal clavicle continues as shown in Table 6). Then similarly, in sequence step 14, treatment of the non-dominant-side lower sacrum and upper ilium would be replaced by application of Logan-Basic Technique pressure by application by the practitioner's finger to the patient's non-dominant-side sacrotuberous ligament (proximal the patient's non-dominant side sacrum). Then in sequence step 15, treatment of the dominant-side upper sacrum and upper ilium would be replaced by application of Logan-Basic Technique pressure by the practitioner's finger to the patient's dominant-side sacrotuberous ligament and/or long dorsal ligament (treatment to the proximal clavicle continues as shown in Table 15).

It is to be understood that the foregoing description is exemplary and explanatory only and is not restrictive of the invention, as disclosed or claimed. Changes and modifications may be made to the disclosed embodiments without departing from the scope of the present invention. These and other changes or modifications are intended to be included within the scope of the present invention, as expressed in the following claims, in the description herein, and in the referenced figures.

What is claimed is:

1. A treatment method comprising:
administering to a patient a sequence of treatment steps in a primary treatment cycle, each of the treatment steps respectively comprising one or more treatment patterns, the treatment patterns respectively comprising one or more physical manipulations of body structures by a health care practitioner executed in a predetermined order;
administering to a patient a sequence of treatment steps in a secondary treatment cycle, each of the treatment steps respectively comprising one or more treatment patterns, the treatment patterns respectively comprising one or more physical manipulations of body structures by a health care practitioner executed in a predetermined order; and
wherein:
the sequence steps of the primary treatment cycle are applied starting from a bottom side of the dominant side of the body and moving toward the patient's upper body non-dominant side; and
the sequence steps of the secondary treatment cycle are applied starting from a bottom side of the non-dominant side of the body and moving toward the patient's upper body dominant side.

2. The method as defined in claim 1, further comprising determining a dominant side of the patient's body.

3. The method as defined in claim 2, wherein determining a dominant side of the patient's body comprises:
positioning the patient in a supine position with legs extended;
alternatively moving the patient's knees towards the patient's chest, and monitoring the range of motion during the movement; and
determining the dominant side of the patient corresponds to the side of the patient where the patient's leg encountered the most restrictive range of motion.

4. The method as defined in claim 1, wherein the sequence of treatment steps in the primary treatment cycle comprises sequential treatment of the patient's dominant-side foot, dominant-side knee, dominant-side hip, dominant-side pubic symphysis, dominant-side lower sacrum and upper ilium; non-dominant-side upper sacrum and upper ilium, non-dominant-side proximal clavicle, non-dominant-side shoulder, non-dominant-side elbow, and non-dominant-side wrist.

5. The method as defined in claim 4, further comprising: prior to the treatment of the patient's non-dominant-side proximal clavicle, treating the patient's dominant-side sacrotuberous ligament with Logan-Basic Technique then treating the patient's non-dominant-side sacrotuberous ligament and/or long dorsal ligament with Logan-Basic Technique.

6. The method as defined in claim 1, wherein the sequence of treatment steps in the secondary treatment cycle comprises sequential treatment of the patient's non-dominant-side foot, non-dominant-side knee, non-dominant-side hip, non-dominant side pubic symphysis, non-dominant-side lower sacrum and upper ilium, non-dominant-side upper sacrum and upper ilium, dominant-side proximal clavicle, dominant-side shoulder, dominant-side elbow, and non-dominant-side wrist.

7. The method as defined in claim 6, further comprising: prior to the treatment of the patient's dominant-side proximal clavicle, treating the patient's non-dominant-side sacrotuberous ligament with Logan-Basic Technique, then treating the patient's dominant-side sacrotuberous ligament and/or long dorsal ligament with Logan-Basic Technique.

8. The method as defined in claim 1, wherein each of the treatment steps of the primary treatment cycle are administered to the patient no more often than one treatment step per day.

9. The method as defined in claim 1, wherein each of the treatment steps of the secondary treatment cycle are administered to the patient no more often than one treatment step per day.

10. The method as defined in claim 1, wherein 9 treatment steps are administered to the patient in the primary treatment cycle and 9 treatment steps are administered to the patient in the secondary treatment cycle.

11. The method as defined in claim 1, wherein a total of 18 treatment steps are administered to the patient.

12. The method as defined in claim 1, the patient's treatment is completed with 18 treatment steps, each step administered on a different calendar day.

13. The method as defined in claim 1, wherein a total of 20 treatment steps are administered to the patient.

14. The method as defined in claim 1, wherein the patient's treatment is completed with 20 treatment steps, each step administered on a different calendar day.

15. The method as defined in claim 1, wherein the patient is treated without applying direct treatment manipulation to one of the cervical spine or the thoracic spine.

16. The method as defined in claim 1, wherein a ninth treatment step is performed on the patient in the same day as a tenth treatment step.

17. The method as defined in claim 1, wherein in the primary treatment cycle, treatments are applied to the patient's non-dominant shoulder/humerus, elbow, and wrist on different calendar days.

18. The method as defined in claim 1, wherein in the secondary treatment cycle, treatments are applied to the patient's dominant shoulder/humerus, elbow, and wrist on different calendar days.

19. A method for treating subluxations in a joint of an organism, the method comprising providing adjustments to structures in a dominant foot of the organism, followed by a non-dominant foot of the organism, whereby improved motility in the foot structures mitigate shock impulses that are coupled to the joint from the feet when the feet strike a hard surface.

20. A method for reducing injuries in a living organism comprising providing adjustments to structures related to a foot and leg of the organism thereby improving flexibility of a public symphysis joint in the organism.
